SEQUENCE LISTING (3 sequences)

SEQ ID NO: 1 - protein, 5 aa, Artificial Sequence
  For exp. 3.3, 3.6, 3.9; synthetic construct
CALAA

SEQ ID NO: 2 - protein, 12 aa, Artificial Sequence
  For exp. 3.4, 3.7, 3.11; synthetic construct
CAANDENYALAA

SEQ ID NO: 3 - protein, 9 aa, Artificial Sequence
  For exp. 3.10; synthetic construct
CDENYALAA

(19) United States
(12) Patent Application Publication
(10) Pub. No.: US 2025/0188117 A1
(43) Pub. Date: Jun. 12, 2025

(54) C-Terminal Peptide Modification

(71) Applicant: Technische Universiteit Delft, Delft (NL)

(72) Inventors: Rienk Eelkema, Delft (NL); Mariano Macchione, Delft (NL); Peggy Bohlaender, Delft (NL)

(21) Appl. No.: 18/282,288

(22) PCT Filed: Mar. 25, 2022
(86) PCT No.: PCT/NL2022/050164

§ 371 Date: Sep. 15, 2023

(30) Foreign Application Priority Data
Mar. 26, 2021 (NL) 2027847

(51) Int. Cl. C07K 1/107; A61K 47/68; C12Q 1/6874

(52) U.S. Cl. CPC C07K 1/1077; A61K 47/6803; C12Q 1/6874

(57) ABSTRACT

A method for providing a cargo to a C-terminal end of a peptide comprises a first stage and a second stage, wherein the first stage comprises reacting the C-terminal end of the peptide with a first reactant in the presence of a first catalyst and first radiation to provide a first intermediate, wherein the first catalyst is configured to decarboxylate the C-terminal end of the peptide in the presence of the first radiation; and wherein the second stage comprises exposing the first intermediate to a second reactant.

### FIELD OF THE INVENTION

The invention relates to a method for providing a cargo to a C-terminal end of a peptide. The invention further relates to a peptide-cargo conjugate. The invention further relates to a peptide array comprising the peptide-cargo conjugate. The invention further relates to a use of the peptide-cargo conjugate.

### BACKGROUND OF THE INVENTION

Methods for modifying the C-terminal end of a protein are known in the art. For instance, Bloom et al, “Decarboxylative Alkylation: An Approach to Site-Selective Bioconjugation of Native Proteins via Oxidation Potentials”, Nature Chemistry, 2018, describes visible-light mediated single-electron transfer as a mechanism towards enabling site- and chemoselective bioconjugation.

Malins, “decarboxylative couplings as versatile tools for late-stage peptide modifications”, Peptide Science, 2018, reviews decarboxylative coupling strategies for targeted functionalization of native peptidic acids.

WO2016196931 describes synthetic methods to produce molecular species through conjugate additions via decarboxylative mechanisms. It describes methods of functionalization of peptide residues, including selective functionalization of peptide C-terminal residues.

### SUMMARY OF THE INVENTION

C-terminal modifications of peptides, especially of proteins, may be important for sensors, arrays, (fundamental) research, sequencing applications, as well as for the synthesis of protein-drug conjugates. However, it may be challenging to selectively modify the C-terminal end of native peptides or proteins.

For instance, prior art methods for C-terminal peptide modification may only be compatible with a small range of C-terminal residues.

Prior art methods may further relate to the synthesis of (recombinant) mutant proteins with functional groups for further modifications. However, such methods may not be compatible with modifying peptides from (natural) biological samples.

Further, prior art methods may require solid phase synthesis methods, or may require the use of non-natural coupling chemistry to attach a cargo to the peptide.

Prior art methods may further require specific solvents, which may not be compatible with peptides insoluble in such solvents, which may be particularly relevant with regards to relatively large peptides and proteins.

In addition, prior art methods may be incompatible with many types of cargo.

Hence, prior art methods may be rather restricted with respect to compatibility with peptides, particularly with regards to C-terminal residues, and different types of cargo.

Hence, it is an aspect of the invention to provide an alternative method for the C-terminal peptide modification, which preferably further at least partly obviates one or more of above-described drawbacks. The present invention may have as object to overcome or ameliorate at least one of the disadvantages of the prior art, or to provide a useful alternative.

In a first aspect, the invention may provide a method for providing a cargo to a C-terminal end of a peptide, i.e., a method for providing a peptide-cargo conjugate. The method may comprise a first stage and a second stage. In embodiments, the first stage may comprise reacting the C-terminal end of the peptide with a first reactant in the presence of a first catalyst and first radiation to provide a first intermediate. In particular, the first catalyst may be configured to decarboxylate the C-terminal end of the peptide in the presence of the first radiation. In embodiments, the first reactant may have a first chemical structure according to formula I:

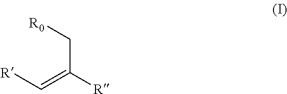

especially wherein R₀ is selected from the group consisting of H, halides, such as Cl, Br, and I, O-acyl groups, such as O(C═O)R groups with R being alkyl or aryl, carbonate groups, such as O(C═O)OR groups with R being alkyl or aryl, and sulfonate groups, such as OS(O₂)-R groups with R being alkyl or aryl, and NR₃ groups, especially wherein each R may be independently selected from H and alkyl groups, or especially wherein NR₃ comprises pyridinium or a derivative thereof; and especially R′ is selected from the group consisting of H, aryl groups and alkyl groups; and especially wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group. In embodiments, the second stage may comprise exposing the first intermediate to a second reactant, especially wherein the second reactant has a second chemical structure according to formula III:

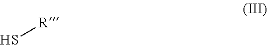

wherein R′″ comprises the cargo.

The method of the invention relates to selectively decarboxylating the C-terminal end of a peptide, which may introduce a radical, and subsequently attaching a reactive group to the C-terminal end of the peptide, especially a thioester group, or especially a Michael acceptor group. The reactive group may then be used in a second stage to attach a cargo to the peptide. In particular, the method of the invention may provide the benefit that a large variety of cargos, including other peptides, can be selectively covalently linked to the C-terminal end of a natural peptide. This may, for instance, be beneficial in the context of peptide sequencing, especially protein sequencing, in forming antibody drug conjugates, and for providing peptide arrays, especially protein arrays.

In particular, the first stage may provide a first intermediate having a thioester functional group arranged at the C-terminal end of the peptide and/or having a Michael acceptor group arranged at the C-terminal end of the peptide, especially a thioester group, or especially a Michael acceptor group. For instance, in embodiments wherein the first intermediate comprises a C-terminal thioester functional group, the first intermediate may react with a second reactant having a second chemical structure according to formula IIIA (see below), such as a second reactant having a cysteine functional group, to attach the cargo to the peptide. In embodiments wherein the first intermediate comprises a C-terminal Michael acceptor group, the first intermediate may especially react with a second reactant having a second chemical structure according to formula III to attach the cargo to the peptide.

The method of the invention may provide a general approach to attach nearly any cargo to the C-terminal end of most peptides, without requiring solid phase methods. Further, the method may be compatible with natural peptides.

Hence, in specific embodiments, the invention provides a method for providing a cargo to a C-terminal end of a peptide, the method comprising a first stage and a second stage, wherein the first stage comprises reacting the C-terminal end of the peptide with a first reactant in the presence of a first catalyst and first radiation to provide a first intermediate, wherein the first catalyst is configured to decarboxylate the C-terminal end of the peptide in the presence of the first radiation, and wherein the first reactant has a first chemical structure according to formula I:

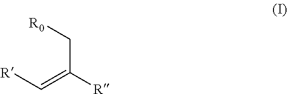

wherein R₀ is selected from the group consisting of H, halides, O-acyl groups, carbonate groups, sulfonate groups and NR₃ groups; wherein R′ is selected from the group consisting of H, aryl groups and alkyl groups; wherein R″ is an electron withdrawing group comprising a functional group selected from the group consisting of an ester, a thioester, an amide, a ketone, a nitro, a sulfoxide, a sulfone, a phosphate ester, and an acylhydrazide, a cyano group, and a trihalogenmethyl group; and wherein the second stage comprises exposing the first intermediate to a second reactant, wherein the second reactant has a second chemical structure according to formula III:

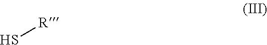

wherein R′″ comprises the cargo.

Hence, the invention may provide a method for providing a cargo to a C-terminal end of a peptide.

The term “cargo” may herein refer to any compound that may be relevant to attach to a peptide. For instance, the cargo may comprise a second peptide, an antibody, a drug, a transcription factor, a purification tag, a nanoparticle, a fluorescent dye, and an anchoring tag.

The term “peptide” may herein refer to a chain of amino acids of any length, especially a chain of at least two amino acids, more especially a chain of ten or more amino acids. The term peptide may thus also refer to an oligopeptide, to a polypeptide, and to a protein. In embodiments, the peptide may comprise a natural peptide, especially a natural protein, i.e., a native peptide (or protein) produced by a naturally occurring organism. The peptide may especially comprise a number of amino acids selected from the range of 2-4000, such as 3-2500, especially 3-1000. In further embodiments, the peptide may comprise at most 500 amino acids, such as at most 100 amino acids, especially at most 50 amino acids, such as at most (about) 40 amino acids. In further embodiments, the peptide may comprise at least 2 amino acids, such as at least 3 amino acids, especially at least 5 amino acids. In further embodiments, the peptide may especially comprise a number of amino acids selected from the range of 2-60, especially from the range of 2-40, such as from the range of 3-40.

The method may especially comprise a first stage and a second stage. The term “stage” and similar terms used herein may refer to a (time) period (also “phase”) of a method. The first stage and the second stage may especially be temporally separated, wherein the second stage is temporally arranged after the first stage.

The first stage may comprise reacting the C-terminal end of the peptide with a first reactant in the presence of a first catalyst and first radiation, especially to provide a first intermediate.

The first catalyst may especially be configured to decarboxylate the C-terminal end of the peptide in the presence of the first radiation. In particular, the first catalyst May comprise a riboflavin catalyst, including riboflavin derivatives, such as riboflavin tetrabutyrate, and an iridium catalyst, such as Ir[dF(CF₃)ppy]₂(dtbbpy)PF₆, especially riboflavin tetrabutyrate. The first catalyst may especially be a photocatalyst decarboxylating the C-terminal end of a peptide when exposed to first radiation. Hence, the first catalyst may be configured to decarboxylate the C-terminal end of the peptide in the presence of the first radiation. The first radiation may especially comprise a first wavelength in the range of 250-600 nm, especially from the range of 300-600 nm. In further embodiments, the first radiation may comprise a first wavelength in the range of 375-525 nm, such as in the range of 400-525 nm. Hence, in embodiments, the first radiation may have an intensity at a first wavelength in the range of 250-600 nm, especially in the range of 300-600 nm. In further embodiments, the first radiation may have an intensity at a first wavelength in the range of 375-525 nm, such as in the range of 400-525 nm. The first radiation may especially comprise a first wavelength selected such that the first catalyst decarboxylates the C-terminal end of the protein when exposed to the first radiation, i.e., the first wavelength may be suitable for the first catalyst to decarboxylate the C-terminal end of the protein when exposed to the first radiation.

In further embodiments, the method may comprise providing the first radiation with a light source, especially a solid state light source, such as an LED. The light source may especially have a peak wavelength in the range of 250-600 nm, especially from the range of 300-600 nm. In further embodiments, the light source may have a peak wavelength in the range of 375-525 nm, such as in the range of 400-525 nm.

In further embodiments, wherein the first solvent comprises, especially consists of, an organic solvent, the first catalyst may comprise an iridium catalyst selected from the group comprising (Ir[dF(CF₃)ppy]₂(dtbpy))PF₆ (CAS Number 870987-63-6), [Ir(dtbbpy)(ppy)₂]PF₆ (CAS Number 676525-77-2), [Ir(dFppy)₂(dtbbpy)]PF₆ (CAS Number 1072067-44-7), [Ir(dFCF₃ppy)₂-(5,5′-dCF₃bpy)]PF₆ (CAS Number 1973375-72-2), [Ir(dF(Me)ppy)₂(dtbbpy)]PF₆ (CAS Number 1335047-34-1), [Ir{dFCF₃ppy}₂(bpy)]PF₆ (CAS Number 1092775-62-6), [Ir(p-F(Me)ppy)₂-(4,4′-dtbbpy)]PF₆ (CAS Number 808142-88-3), Ir(dFppy)₃ (CAS Number 387859-70-3), Ir(dFFppy)₂(dtbbpy)PF₆ (CAS Number 2042201-18-1), and Ir[dFFppy]₂-(4,4′-dCF₃bpy)PF₆ (CAS Number 2030437-92-2). In such embodiments, the first environment, especially the first solvent, may especially comprise 0.005-2 mM of the first catalyst, such as 0.01-1 mM.

In further embodiments, wherein the first solvent comprises water, or wherein the first solvent comprises an organic solvent/water mixture, the first catalyst may comprise a riboflavin catalyst selected from the group comprising riboflavin tetrabutyrate (CAS Number 752-56-7), Lumiflavin (CAS Number 1088-56-8), Riboflavin (CAS Number 83-88-5), Riboflavin 5′-monophosphate sodium salt (CAS Number 130-40-5), Lumichrome (CAS Number 1086-80-2), riboflavin N-ethyl carbamate, 4-[2-(7,8-dimethyl-2,4-dioxo-3,4-dihydro-benzo[g]pteridin-10(2H)-yl)ethoxy]-4-oxobutanoic acid, 10-ethyl-3,7,8-trimethyl-benzo[g]-pteridine-2,4(3H, 10H)-dione, 7,8-dimethyl-10-{2-[bis(2-hydroxyethyl)amino]ethyl}isoalloxazine hydrochloride, and 7,8-diethyl-10-(1′-d-ribityl)isoalloxazine. In such embodiments, the first environment, especially the first solvent, may especially comprise 0.005-600 mM of the first catalyst, such as 0.01-300 mM.

In further embodiments, the first catalyst may comprise a mesityl acridinium catalyst. In such embodiments, the first environment, especially the first solvent, may especially comprise 0.01-1 mM of the first catalyst.

The term “iridium catalyst” may herein especially refer to a catalyst comprising iridium, i.e., to an iridium comprising catalyst.

The first reactant may, in embodiments, have a first chemical structure according to formula I:

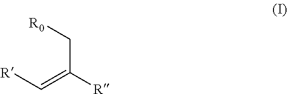

In embodiments, R₀ may be selected from the group comprising H, halides, O-acyl groups, carbonate groups, sulfonate groups and NR₃ groups. In further embodiments, R₀ may comprise H. In further embodiments R₀ may comprise a halide, especially Cl, Br, or I. In further embodiments, R₀ may comprise an O-acyl group, i.e., an O(C═O)R group, wherein R comprises an alkyl or aryl group. In further embodiments, R₀ may comprise a carbonate group, i.e., an O(C═O)OR group, wherein R comprises an alkyl or aryl group. In further embodiments, R₀ may comprise a sulfonate group, such as tosylate and mesylate, especially —OS(O₂)-R wherein R comprises an alkyl or aryl group. In further embodiments, R₀ may comprise an NR₃ group, especially pyridinium or a derivative thereof, or especially wherein each R is independently selected from the group comprising H and alkyl groups.

In further embodiments, R′ may be selected from the group consisting of H, aryl groups and alkyl groups. In further embodiments, R′ may be H. In further embodiments, R′may comprise an aryl group. In further embodiments, R′ may comprise an alkyl group.

In further embodiments, R″ may be an electron withdrawing group, especially an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group. In particular, the electron withdrawing group may be selected such that the depicted C═C bond in formula I may be (more) reactive with respect to the decarboxylated C-terminal end of the peptide. In further embodiments, the electron withdrawing group may especially comprise a functional group selected from the group consisting of an ester group and a thioester group, especially an ester group, or especially a thioester group. In further embodiments, the electron withdrawing group may especially comprise a ketone functional group. In further embodiments, the electron withdrawing group may especially comprise a nitro functional group. In further embodiments, the electron withdrawing group may especially comprise a sulfoxide functional group. In further embodiments, the electron withdrawing group may especially comprise a phosphate ester functional group. In further embodiments, the electron withdrawing group may especially comprise an acylhydrazide functional group. In further embodiments, the electron withdrawing group may especially comprise a cyano functional group. In further embodiments, the electron withdrawing group may especially comprise a trihalogenmethyl functional group, especially wherein the trihalogenmethyl functional group comprises halogens selected from the group comprising F, Cl and Br, more especially wherein the trihalogenmethyl functional group comprises three identical halogens, such as a trihalogenmethyl functional group comprising —CF₃, —CCl₃ or CBr₃, especially —CF₃.

In further embodiments R″ may comprise a functional group selected from the group consisting of an ester, a cyano group, and —CF₃. In embodiments wherein R″ comprises an ester, R″ may especially comprise a methylester. Hence, in embodiment, R″ may comprise a methylester.

The term “alkyl group” may herein especially refer to an alkyl group comprising 1-10 C atoms, especially 1-6 C atoms, such as 1-4 C atoms.

The term “aryl group” may herein especially refer to a functional group or substituent derived from an aromatic ring comprising 5-10 elements in the aromatic ring.

During the first stage, the peptide may, in embodiments, be exposed to a first environment. In particular, the peptide (and the first catalyst) may be arranged in a first mixture comprising a first solvent. In embodiments, the first solvent may especially be selected from the group comprising water, dimethyl sulfoxide (DMSO), dimethylformamide (DMF), acetonitrile, dimethylacetamide, N-methylpyrrolidone, and tetrahydrofuran, especially from the group comprising water, DMSO and DMF.

The first mixture may especially comprise one or more of the first solvent, the peptide, and the first reactant. In embodiments, the first mixture may comprise further additives (see below).

In embodiments, the first solvent may comprise water. In such embodiments, the first solvent may have a pH selected from the range of 1-11, especially from the range of 1.5-8, or from the range of 7-10. Hence, in embodiments, the first environment may have a pH selected from the range of 1-11, especially from the range of 1.5-8, such as from the range of 7-10. In particular, in embodiments wherein the first reactant has a first chemical structure according to formula IA it may be particularly beneficial for the first stage to occur in a first environment with a pH in the range of 5-10, such as about 7.

In further embodiments, the first solvent may have a pH of at least 1, especially at least 1.5, such as at least 2. In further embodiments, the first environment, especially the first solvent, may have a pH of at least 5, especially at least 6, such as at least 7. In further embodiments, the first environment, especially the first solvent, may have a pH of at most 11, especially at most 10.5, such as at most 10. In further embodiments, the first environment, especially the first solvent, may have a pH of at most 9, especially at most 8.5, such as at most 8.

In embodiments, the first solvent may comprise at least 70 vol. % water, such as at least 80 vol. %, especially at least 90 vol. %, including 100 vol. %.

In further embodiments, the first solvent may comprise an organic solvent, especially an organic solvent selected from the group comprising DMSO, acetonitrile, and DMF. In embodiments, the first solvent may comprise at least 70 vol. % of the organic solvent, such as at least 80 vol. %, especially at least 90 vol. %, including 100 vol. %. The term “organic solvent” may herein also refer to a plurality of (different) organic solvents.

The first environment, especially the first mixture, more especially the first solvent, may, in embodiments, have a first temperature selected from the range of 5-45° C., especially from the range of 10-40° C. In further embodiments, the method may comprise controlling the first temperature, especially during the first stage, to be at most 45° C., such as at most 40° C. In further embodiments, the method may comprise controlling the first temperature using a temperate control element, especially a cooling element.

In embodiments, especially in embodiments wherein the first solvent comprises water, the first mixture may further comprise a sequestering agent for reactive oxygen species. In particular, in embodiments, the first mixture may comprise 1-10 vol. % glycerol.

Further, in embodiments, the first solvent may comprise water and an organic solvent, such as DMF or 1-butanol. In particular, the first solvent may comprise at least 50 wt. % water and 2-20 wt. % of the organic solvent, such as 5-15 wt. %. The organic solvent may facilitate dissolving of the first catalyst. In further embodiments, the first solvent may comprise a water phase and an organic phase.

In embodiments, the first environment, especially the first mixture, may comprise 0.02-100 mM of the peptide, especially 0.05-50 mM of the peptide.

In further embodiments, the first environment, especially the first mixture, may comprise 0.05-1000 mM of the first reactant, especially 0.1-500 mM.

In embodiments, the first environment, especially the first mixture, may comprise a salt, especially 0.05-100 mM of a salt, such as 1-50 mM of the salt, especially 2-10 mM of the salt. In embodiments, especially embodiments wherein the first solvent comprises an organic solvent, the salt may be selected from the group comprising phosphates, hydrogen phosphates, dihydrogen phosphates, formates, trifluoroacetates, acetates, fluorides and carbonates, especially from the group comprising K₂HPO₄, cesium formate, sodium trifluoroacetate, CsF, CsOAc, Cs₂CO₃, and K₂CO₃, more especially from the group comprising K₂HPO₄, and cesium formate.

Following the first stage, there may still be leftover first reactant in the first mixture. In specific cases, the leftover first reactant may, for instance when storing the first mixture at the end of the first stage, undesirably react with the peptide. Hence, in embodiments, the method may comprise adding a thiol compound after (or: “at the end”) of the first stage, especially a thiol compound selected from the group comprising 3-Mercapto-N-methylpropanamide, 2-mercaptoethanol and ethyl 3-mercaptopropionate. The addition of the thiol compound may reduce, especially eliminate, by-product formation.

The second stage may comprise exposing the first intermediate to a second reactant. In particular, the second stage may comprise reacting the first intermediate and the second reactant to provide a peptide-cargo conjugate. The second reactant may especially have a second chemical structure according to formula III:

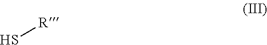

wherein R′″ comprises the cargo.

In embodiments, during the second stage, the first intermediate may be exposed to a second environment. In particular, the first intermediate (and the second reactant) may be arranged in a second mixture comprising a second solvent. In embodiments, the second solvent may especially be selected from the group comprising water, DMSO, DMF, acetonitrile, dimethylacetamide, N-methylpyrrolidone, and tetrahydrofuran, especially from the group comprising water, DMSO, acetonitrile and DMF.

The second mixture may especially comprise one or more of the second solvent, the first intermediate, and the second reactant. In embodiments, the second mixture may comprise further additives (see below).

In embodiments, the second solvent may comprise water. In such embodiments, the second solvent may have a pH selected from the range of 5.5-10.5, especially from the range of 6-10, such as from the range of 6.5-9.5. In further embodiments, the second solvent, may have a pH of at least 4.5, especially at least 5, such as at least 5.5. In further embodiments, the second solvent may have a pH of at least 6, especially at least 6.5. In further embodiments, the second solvent may have a pH of at most 11, especially at most 10.5. In further embodiments, the second solvent, may have a pH of at most 10, especially at most 9.5.

With regards to first intermediates having a chemical structure according to formula IIB (see below), a high pH may lead to by-production formation, whereas a low pH may reduce reaction rate and yield. In particular, in such embodiments, the second solvent may have a pH selected from the pH range of 6-10, such as a pH of about 8. Such pH values for the second solvent may yield particularly good results.

During the second stage, the second environment, especially the second mixture, more especially the second solvent, may, in embodiments, have a second temperature. If the second temperature is low, the reaction between the first intermediate may progress slowly. However, higher second temperatures may lead to (more) by-product formation. Hence, in embodiments, the second temperature may be selected from the range of 2-55° C., such as from the range of 4-50° C. In embodiments where the first intermediate has a first chemical structure according to formula IIA (see below), the second temperature may especially be selected from the range of 30-50° C. In embodiments where the first intermediate has a first chemical structure according to formula IIB (see below), the second temperature may especially be selected from the range of 4-45° C.

In further embodiments, the second solvent may comprise an organic solvent, especially an organic solvent selected from the group comprising dimethyl sulfoxide (DMSO), acetonitrile, and dimethylformamide (DMF).

In embodiments, the second solvent may comprise water and an organic solvent, especially wherein the second solvent comprises at most 25 vol. % organic solvent, such as at most 20 vol. %. In further embodiments, the second solvent may comprise at least 70 vol. % water, such as at least 75 vol. % water, especially at least 80 vol. % water.

In embodiments, the second environment, especially the second mixture, may comprise 0.005-200 mM of the first intermediate, especially 0.01-100 mM of the first intermediate, such as 0.1-100 mM of the first intermediate.

In further embodiments, the second environment, especially the second mixture, may comprise 0.005-400 mM of the second reactant, especially 0.01-200 mM, such as 0.1-200 mM.

The presence of oxygen may be detrimental to providing the first intermediate in the first stage and/or to providing the protein-cargo conjugate in the second stage. In particular, the presence of oxygen may be detrimental for the photocatalyzed decarboxylation of the peptide in the first stage. Specifically, in the first stage, the presence of oxygen may result in a reduced yield of the first intermediate. Further, in the second stage, the presence of oxygen can cause oxidation of SH groups that may be present in the second reactant or in a reducing agent (see below), which can result in disulfide formation. Hence, in both stages, the presence of oxygen may hamper the method of the invention.

Hence, in embodiments, the first environment, especially the first mixture, may comprise a degassed buffer. In particular, the first environment, especially the first mixture may comprise ≤10 ppm dissolved oxygen, such as ≤5 ppm dissolved oxygen, especially ≤2 ppm dissolved oxygen. In further embodiments, the first environment, especially the first mixture, may comprise ≤1 ppm dissolved oxygen, such as ≤0.8 ppm dissolved oxygen, especially ≤0.6 ppm dissolved oxygen, such as ≤0.5 ppm dissolved oxygen. In further embodiments, the first mixture may comprise at most 0.4 ppm dissolved oxygen, such as 0.2-0.4 ppm dissolved oxygen.

In embodiments, the method may comprise controlling, especially reducing, the (dissolved) oxygen concentration in the first environment, especially in the first mixture. In particular, the method may comprise sparging (or “purging”) the first mixture, especially the first solvent, with an inert gas, especially with N₂.

In further embodiments, the method may comprise executing the first stage in a degassed buffer under inert gas, especially N₂, or especially Ar. Argon may be particularly suitable as it is heavier than air.

In further embodiments, the second environment, especially the second mixture, may comprise a degassed buffer. In particular, the second environment, especially the second mixture, may comprise ≤10 ppm dissolved oxygen, such as ≤5 ppm dissolved oxygen, especially ≤2 ppm dissolved oxygen. In further embodiments, the second environment, especially the second mixture, may comprise ≤1 ppm dissolved oxygen, such as ≤0.8 ppm dissolved oxygen, especially ≤0.6 ppm dissolved oxygen. In further embodiments, the second mixture may comprise at most 0.4 ppm dissolved oxygen, such as 0.2-0.4 ppm dissolved oxygen.

As will be known to the person skilled in the art, dissolved oxygen may especially be determined using a Winkler titration. In particular, especially with regards to water, ppm and mg/L may typically be used interchangeably with regards to dissolved oxygen. Hence, in embodiments, the first mixture (or the second mixture) may comprise ≤10 mg/L dissolved oxygen, such as ≤5 mg/L dissolved oxygen, especially ≤2 mg/L dissolved oxygen. In further embodiments, the first mixture (or the second mixture) may comprise ≤1 mg/L dissolved oxygen, such as ≤0.8 mg/L dissolved oxygen, especially ≤0.6 mg/L dissolved oxygen, such as ≤0.5 mg/L dissolved oxygen. In further embodiments, the first mixture (or the second mixture) may comprise at most 0.4 mg/L dissolved oxygen, such as 0.2-0.4 mg/L dissolved oxygen.

In embodiments, the method may comprise controlling, especially reducing, the (dissolved) oxygen concentration in the second environment, especially in the second mixture. In particular, the method may comprise sparging the second mixture, especially the second solvent, with an inert gas, especially with N₂.

In further embodiments, the method may comprise executing the second stage in a degassed buffer under inert gas, especially N₂, or especially Ar. Argon may be particularly suitable as it is heavier than air.

The first stage and the second stage may especially be performed in (essentially) the same solvent. For example, both the first stage and the second stage may be executed in water. However, in embodiments, the first stage and the second stage may be executed in different solvents. In such embodiments, the method may further comprise an intermediate stage. The intermediate stage may comprise separating the first intermediate, especially from remainder of the first reactant and/or the peptide and/or the first catalyst, especially from the first catalyst, or especially from (remainder of) the first reactant, or especially from (remainder of) the peptide. The intermediate stage may further comprise separating the first intermediate from the first solvent. In embodiments, the intermediate stage may comprise an extraction, especially an extraction with an organic solvent, such as an extraction with one or more of ethyl acetate, dichloromethane, or an ether, such as with an ether selected from the group comprising diethyl ether, methyl tert-butyl ether, and diisopropyl ether.

The intermediate stage may especially be temporally arranged between the first stage and the second stage.

The method of the invention may be compatible with a large variety of C-terminal residues, including (naturally) proteinogenic amino acids, such as canonical amino acids, as well as natural amino acids not naturally incorporated into proteins, such as ornithine, as well as non-natural amino acids. In particular, in embodiments the peptide may comprise a C-terminal residue selected from the group comprising alanine, arginine, asparagine, aspartate, cysteine, glutamate, glutamine, histidine, leucine, lysine, methionine, phenylalanine, proline, pyrrolysine, selenocysteine, tryptophan, tyrosine, and ornithine, especially from the group comprising alanine, arginine, asparagine, aspartate, cysteine, glutamate, glutamine, histidine, leucine, lysine, methionine, phenylalanine, proline, tryptophan, and tyrosine.

In particular, in embodiments wherein the first reactant has a first chemical structure according to formula IA, the peptide may comprise a C-terminal residue selected from the group comprising alanine, arginine, asparagine, aspartate, cysteine, glutamate, glutamine, histidine, leucine, lysine, methionine, phenylalanine, proline, pyrrolysine, selenocysteine, tryptophan, tyrosine, and ornithine, especially from the group comprising alanine, arginine, asparagine, aspartate, cysteine, glutamate, glutamine, histidine, leucine, lysine, methionine, phenylalanine, proline, tryptophan, and tyrosine.

In further embodiments, wherein the first reactant has a first chemical structure according to formula IB, the peptide may comprise a C-terminal residue selected from the group comprising alanine, arginine, asparagine, aspartate, cysteine, glutamate, glutamine, histidine, leucine, lysine, methionine, phenylalanine, proline, pyrrolysine, selenocysteine, tryptophan, tyrosine, and ornithine, especially from the group comprising alanine, arginine, asparagine, aspartate, cysteine, glutamate, glutamine, histidine, leucine, lysine, methionine, phenylalanine, proline, tryptophan, and tyrosine. In particular, a cargo had been successfully attached using the method of the invention and a first intermediate according to formula IIB for peptides with the C-terminal residues asparagine, arginine, and proline. Hence, in further embodiments, the peptide may comprise a C-terminal residue selected from the group comprising asparagine, arginine, and proline.

For some amino acids in the peptide, such as for cysteine and selenocysteine, it may be beneficial to protect the amino acids prior to the first stage. Hence, in embodiments, the method may further comprise a preparation stage, temporally arranged before the first stage, wherein the preparation stage comprises providing a protecting group to amino acids of a predefined amino acid type, such as cysteine and/or selenocysteine. For instance, in embodiments, the preparation stage may comprise reacting the peptide with acrylamide, maleimide, alpha-halo acetamide. In further embodiments, the method may comprise capping cysteine and/or selenocysteine by disulfide formation with a small molecule activated disulfide.

In embodiments, the first reactant may have a first chemical structure according to formula I:

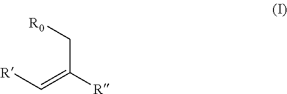

especially wherein R₀ is H, and especially wherein R′ is an alkyl group, and especially wherein R″ is a thioester. Hence, in embodiments the first reactant may have a first chemical structure according to formula IA:

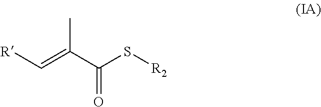

especially wherein R′is an alkyl group, such as a methyl group, and especially wherein R₂ is an alkyl group. In embodiments, R₂ may comprise an alkyl group. In further embodiments, R₂ may optionally have additional functionalities down the chain, especially a functionality selected from the group comprising amide, ester, ether, alcohol, aldehyde, carboxylic acid, amine, aromatic.

In embodiments wherein the first reactant has a first chemical structure according to formula IA, the first catalyst may especially comprise an iridium catalyst. Iridium catalysts may work particularly well in combination with such first reactants.

Similarly, in embodiments wherein the first reactant has a first chemical structure according to formula IA, the first solvent may especially comprise DMSO, and the second solvent may especially comprise water.

Such embodiments may be particularly suitable for providing a thioester functional group to the C-terminal end of the peptide. In further embodiments, the first intermediate may have a chemical structure according to formula IIA:

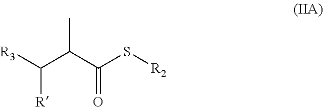

especially wherein R′is an alkyl group, especially wherein R₂ is an alkyl group, and especially wherein R₃ refers to the peptide.

In further embodiments, the first solvent may especially comprise an anhydrous solvent, such as one or more of DMSO and DMF, i.e., in embodiments, the first stage may especially be executed in a first solvent, wherein the first solvent comprises an anhydrous solvent, such as one or more of DMSO and DMF. In particular, the anhydrous solvent may improve the stability of the first intermediate.

In further embodiments, the second reactant may especially have a second chemical structure according to formula IIIA:

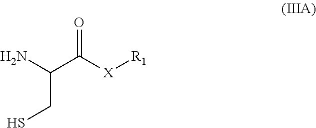

especially wherein X is O or NH, and especially wherein R1 comprises the cargo.

In particular, in embodiments, the method, especially the second stage, may comprise reacting the first intermediate having a chemical structure according to formula IIA with a second reactant having a chemical structure according to formula IIIA. In particular, the first intermediate and the second reactant may react via a second reaction involving a transthioesterification and a subsequent S—N acyl shift.

In embodiments wherein the first intermediate has a chemical structure according to formula IIA, the second environment, especially the second solvent, may especially comprise 0.1-100 mM of the first intermediate. Further, in such embodiments, the second environment, especially the second solvent, may comprise 0.1-200 mM of the second reactant.

In embodiments wherein the first intermediate has a chemical structure according to formula IIA, the second stage may essentially be executed according to a Native Chemical Ligation (NCL) procedure, as described in Agouridas et al., “Native Chemical Ligation and Extended Methods: Mechanisms, Catalysis, Scope, and Limitations”, 2019, Chemical Reviews, and in Boll et al., “One-pot chemical synthesis of small ubiquitin-like modifier protein-peptide conjugates using bis(2-sulfanylethyl)amido peptide latent thioester surrogates”, 2015, Nature Protocols, which are hereby herein incorporated by reference. Hence, in embodiments, the second mixture may comprise 4-Mercaptophenylacetic acid (MPAA); the presence of MPAA may facilitate the formation of a reactive thioester intermediate, which may beneficially affect the second stage. Similarly, in embodiments, the second mixture may comprise tris(2-carboxyethyl)phosphine hydrochloride (TCEP); the presence of TCEP may facilitate maintaining free SH groups on the first intermediate and/or the second reactant, which may beneficially affect the second stage. It will be clear to the person skilled in the art that further additives may be used to beneficially affect an NCL procedure, which may in turn beneficially affect the second stage of embodiments of the method of the invention.

Hence, in embodiments, the method may provide a peptide-cargo conjugate having a chemical structure according to formula IVA:

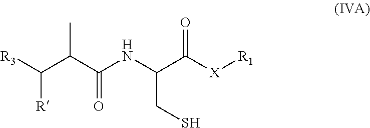

especially wherein R′ is an alkyl group; especially wherein R₃ refers to the peptide; especially wherein X is O or NR, wherein R is H or an alkyl, especially wherein X is O or NH; and especially wherein R₁ comprises the cargo.

In particular, the second chemical structure according to formula IIIA may resemble an N-terminal cysteine residue, optionally bound to the cargo via an amide (CONR), especially CONH, such as in a natural protein with an N-terminal cysteine residue, or optionally bound to the cargo via an ester (COO). Such embodiments may be particularly convenient for attaching a cargo comprising a second peptide, wherein the second peptide has an N-terminal cysteine residue.

Hence, in embodiments, the cargo may comprise a second peptide. The peptide-cargo conjugate may, thus, in embodiments, comprise a peptide-peptide conjugate, i.e., a fusion peptide. In further embodiments, the second peptide may have an N-terminal end according to formula IIIA, especially an N-terminal cysteine residue. Hence, the method of the invention may facilitate providing fusion peptides (or proteins), especially wherein the peptide, and optionally the second peptide, is naturally produced. Hence, the method of the invention may facilitate providing a non-recombinant fusion peptide (or protein).

It will be clear to the person skilled in the art that R₃ (see e.g., formula IB) does not comprise the “full” peptide; R₃ may refer to a peptide part that was formerly associated to its C-terminal carboxylic acid group. Specifically, R₃ may comprise the peptide except for the C-terminal carboxylic acid group that was removed via the photodecarboxylation in stage 1. Hence, R₃ may effectively refer to the peptide (or to the peptide part in the conjugate).

In embodiments, R₀ may be selected from the group comprising halides, O-acyl groups, carbonate groups, sulfonate groups and NR₃, and R′ may especially be H. In particular, R₀ may be a “leaving group” facilitating a reaction with the decarboxylated peptide. Hence, in embodiments, the first reactant may have a first chemical structure according to formula IB:

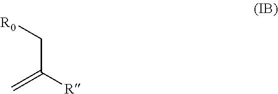

wherein R₀ is selected from the group consisting of halides, O-acyl groups, carbonate groups, sulfonate groups and NR₃ groups; and wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group. In further embodiments, R″ may especially comprise an ester, such as a methylester.

Particularly good results were obtained with a first reactant for which R″ comprises a methylester, especially CH₃O(C═O), and wherein R₀ comprises an O-acyl group, especially O(C═O)CH₃. Hence, in further embodiments, R″ may comprise a methylester, especially CH₃O(C═O). In further embodiments, R₀ may comprise an O-acyl group, especially O(C═O)CH₃.

In embodiments wherein the first reactant has a first chemical structure according to formula IB, the first catalyst may especially comprise riboflavin or a derivative thereof. Riboflavin-based catalysts may work particularly well in combination with such first reactants.

Similarly, in embodiments wherein the first reactant has a first chemical structure according to formula IB, the first solvent may especially comprise water, and the second solvent may especially comprise water.

Further, embodiments wherein the first reactant has a first chemical structure according to formula IB may be particularly suitable for providing a Michael Acceptor functional group to the C-terminal end of the peptide. In further embodiments, the first intermediate may have a chemical structure according to formula IIB:

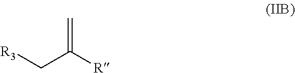

especially wherein R₃ refers to the peptide, and wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group.

In particular, in embodiments, the method, especially the second stage, may comprise reacting the first intermediate having a chemical structure according to formula IIB with a second reactant having a chemical structure according to formula III. In particular, the first intermediate and the second reactant may react via a Michael reaction.

In embodiments wherein the first intermediate has a chemical structure according to formula IIB, the second environment, especially the second mixture, may especially comprise 0.01-100 mM of the first intermediate. Further, in such embodiments, the second environment, especially the second mixture, may comprise 0.01-200 mM of the second reactant.

Hence, in embodiments, the method may provide a peptide-cargo conjugate having a chemical structure according to formula IVB:

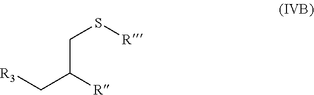

wherein R₃ refers to the peptide, wherein R″ is an electron withdrawing group comprising a functional group selected from the group consisting of an ester, a thioester, an amide, a ketone, a nitro, a sulfoxide, a sulfone, a phosphate ester, and an acylhydrazide, a cyano group, and a trihalogenmethyl group, and wherein R″ comprises the cargo.

In further embodiments, the cargo may comprise an antibody. In particular, as will be known to the skilled person, antibodies may comprise a plurality of polypeptides, and may thus be attached to the peptide as a cargo (similarly to other peptides). Especially, the antibody may be configured to target a (specific) target location in an animal body, especially in a human body, and the peptide may have a (relevant) medical or therapeutic function at the target location. Thereby, the peptide-cargo conjugate may facilitate providing the peptide to a specific site in an animal body, especially in a human body. Hence, the method of the invention may facilitate providing a peptide-antibody conjugate, especially an antibody-drug conjugate.

In embodiments, the cargo may comprise a tag suitable for selectively arranging a (resulting) peptide-cargo conjugate on a target location.

Various specific examples of cargos are described herein. It will be clear to the person skilled in the art, however, that the invention is not limited to such examples, but may further cover a large variety of other cargo compounds.

In a further aspect, the invention may provide a peptide-cargo conjugate, especially a peptide-cargo conjugate obtainable with the method of the invention. In particular, the peptide-cargo conjugate may have a chemical structure according to formula IVA or according to formula IVB.

In embodiments, the peptide-cargo conjugate may have a chemical structure according to formula IVA:

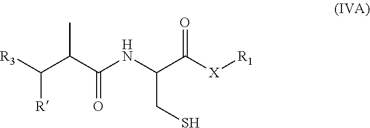

especially wherein R′ is an alkyl group; especially wherein R₃ refers to the peptide; especially wherein X is O or NH, and especially wherein R1 comprises the cargo.

In further embodiments, the peptide-cargo conjugate may have a chemical structure according to formula IVB:

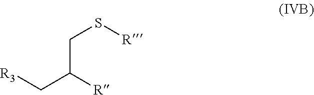

especially wherein R″ is an electron withdrawing group comprising a functional group selected from the group consisting of an ester, a thioester, an amide, a ketone, a nitro, a sulfoxide, a sulfone, a phosphate ester, and an acylhydrazide; especially wherein R′″ comprises the cargo; and especially wherein R₃ refers to the peptide.

In embodiments, the cargo may comprise an antibody. Hence, the peptide-cargo conjugate may comprise a peptide-antibody conjugate.

In further embodiments, the peptide may comprise (one or more polypeptides of) an antibody. In such embodiments, the cargo may, for instance, comprise a drug, such as a peptide drug. In particular, the C-terminus of an antibody may typically not be involved in guest binding and therefore its functionalization may not interfere with antibody binding. As antibodies generally have 4 C-termini, well-defined cargo loading may be facilitated.

In embodiments, the peptide-cargo conjugate may be for use as an antibody drug conjugate. In particular, the peptide-cargo conjugate may be for use as a medicament, especially wherein the peptide has a medically relevant activity, and especially wherein the cargo comprises an antibody. Antibody-drug conjugates (ADCs) are constructs of an antibody linked with a (cytotoxic) drug, especially via a covalent linker. The purpose of the antibody may be (selective) targeting, and the drug may, for instance, serve to kill tumor (or other) cells. The linker may serve to keep the drug attached to the antibody, such as to avoid releasing the drug in the absence of a target cell or tissue. It may thus be important that the drug is attached via a stable link and in a controllable number on the antibody (which may make attachment to the numerous amine or carboxylate side chains on the antibody less appealing as this may result in heterogenous mixtures and may alter the properties of the antibody). Hence, also considering that the C-terminus of an antibody may typically not be involved in guest binding and therefore its functionalization may not interfere with antibody binding, the method of invention may be particularly suitable for providing antibody-drug conjugates.

In embodiments, the cargo may comprise a second peptide. Hence, the peptide-cargo conjugate may comprise a fusion peptide, especially a non-recombinant fusion peptide.

In further embodiments, the cargo may comprise a tag suitable for selectively arranging the peptide-cargo conjugate on a target location, such as on a surface. In further embodiments, the tag may comprise an antibody. Such embodiments may facilitate sequencing of the peptide, and may facilitate providing a peptide array.

Hence, in a further aspect, the invention may provide a peptide array comprising the peptide-cargo conjugate according to the invention, especially wherein the peptide-cargo conjugate comprises a tag. In particular, peptide arrays may comprise libraries of peptides attached to a solid surface such as a glass slide. Such arrays may be used extensively for the identification of peptide drug candidates, enzyme inhibitors, and enzyme substrates, for profiling antibodies and mapping epitopes and receptor-ligand interactions. The most common method for making these arrays is through covalent attachment, which may require reliable and selective attachment chemistry, such as described in Szymczak et al., “Peptide Arrays: Development and Application”, Analytical chemistry, 2018, which is hereby incorporated by reference. Relevant chemical groups are usually introduced in peptide synthesis but may be challenging to introduce in native peptides (peptides isolated from biological sources) with prior art methods. The method of invention, however, may facilitate selective C-terminal introduction of a suitable chemical group in a peptide to facilitate providing a peptide array. Hence, in embodiments, the cargo may comprise a tag suitable for selectively arranging the peptide-cargo conjugate on a target location, especially on a surface. The tag may, for instance, be suitable for click chemistry. In embodiments, the tag may be selected from the group comprising a polynucleotide (configured to hybridize with a complementary polynucleotide at the target location), a biotin tag (suitable to hybridize with avidin or streptavidin at the target location), and an azide-containing peptide (suitable to be chemically immobilized through click chemistry onto a cyclootyne-modified surface). It will be clear to the person skilled in the art that many specific choices may be made for the cargo to enable arranging the peptide-cargo conjugate at a target location of a peptide array.

In a further aspect, the invention may provide a use of the peptide-cargo conjugate for peptide sequencing. In particular, next generation peptide/protein sequencing techniques, such as described in Alfaro et al., “The emerging landscape of single-molecule protein sequencing technologies”, Nature Methods, 2021, which is hereby herein incorporated by reference, may involve either immobilization of the peptide on a surface, or linking of the peptide to a molecular unit that will facilitate translocation through a nanopore. For both scenarios, it may be important that a connection is made in a single, specific location on the peptide, to provide reliable orientation on the surface or when passing through the nanopore. As will be clear to the person skilled in the art, a connection to either the N-terminus or the C-terminus of the peptide may be particularly convenient. The method of the invention may facilitate providing a connection between the peptide and the surface or the molecular unit at the C-terminal end of the peptide, thereby facilitating peptide sequencing of the peptide. In particular, in such embodiments the cargo may comprise a polynucleotide, especially a single stranded polynucleotide. The polynucleotide may be configured for arranging the peptide on a surface, such as via hybridization with a (complementary) polynucleotide attached to the surface. The polynucleotide may further be configured to facilitate passing the peptide-cargo conjugate through a nanopore.

The embodiments described herein are not limited to a single aspect of the invention. For example, an embodiment describing the method may further relate to the (resulting) peptide-cargo conjugate. Similarly, an embodiment of the peptide-cargo conjugate may further relate to embodiments of the method. In particular, an embodiment of the method describing structural formulae with specific groups (e.g., a specific selection of possible R″ groups) may indicate that the peptide-cargo conjugate may, in embodiments, have such groups (such as the specific selection of possible R″ groups).

### BRIEF DESCRIPTION OF THE DRAWINGS

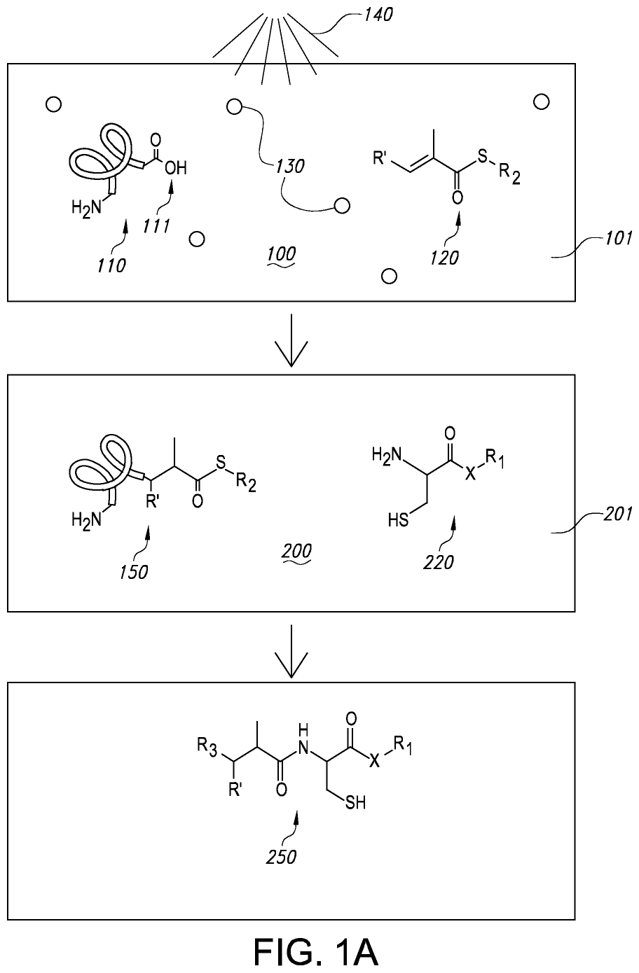

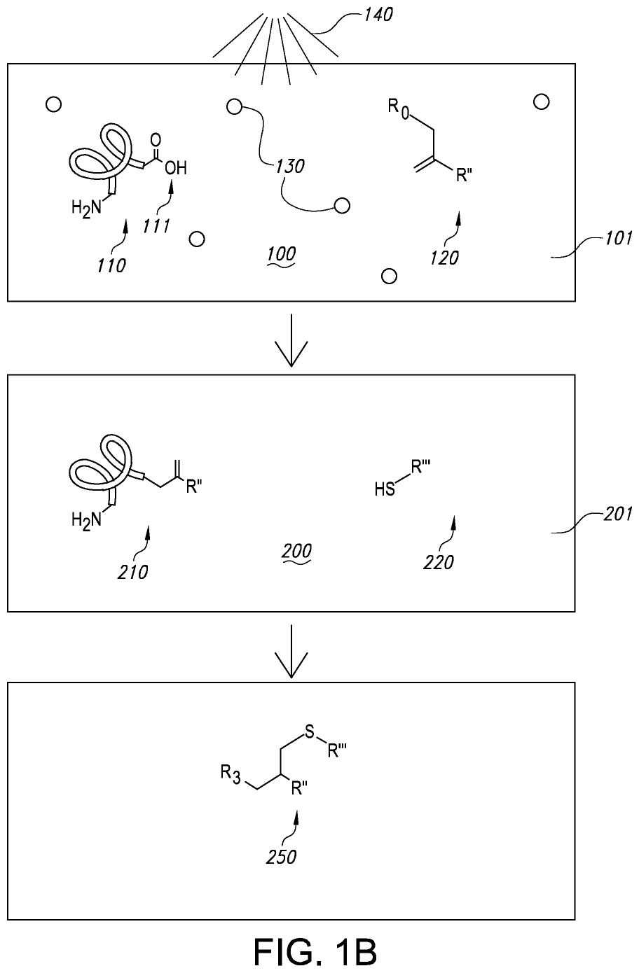

Embodiments of the invention will now be described, by way of example only, with reference to the accompanying schematic drawings in which corresponding reference symbols indicate corresponding parts, and in which: FIG. 1A-B schematically depict embodiments of the method of the invention. The schematic drawings are not necessarily on scale.

### DETAILED DESCRIPTION OF THE EMBODIMENTS

FIG. 1A-B schematically depict embodiments of the method for providing a cargo to a C-terminal end 111 of a peptide 110, i.e., for providing a peptide-cargo conjugate 250. In the depicted embodiments, the method comprises a first stage and a second stage. The first stage may comprise reacting the C-terminal end 111 of the peptide 110 with a first reactant 120 in the presence of a first catalyst 130 and first radiation 140 to provide a first intermediate 150. In particular, the first catalyst 130 may be configured to decarboxylate the C-terminal end 111 of the peptide 110 in the presence of the first radiation 140, which may result in a reactive radical group at the C-terminal end of the peptide. The first reactant 120 may especially have a first chemical structure according to formula I:

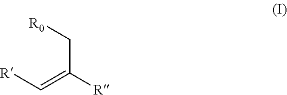

Especially wherein R₀ is selected from the group consisting of H, halides, O-acyl groups, carbonate groups, sulfonate groups, and NR₃ groups, wherein each R may be independently selected from H and alkyl groups, or wherein NR₃ comprises pyridinium or a derivative thereof; especially wherein R′is selected from the group consisting of H, aryl groups and alkyl groups; and especially wherein R″ is an electron withdrawing group comprising a functional group selected from the group consisting of an ester, a thioester, an amide, a ketone, a nitro, a sulfoxide, a sulfone, a phosphate ester, and an acylhydrazide, a cyano group, and a trihalogenmethyl group. In embodiments, the second stage may comprise exposing the first intermediate 150 to a second reactant 220, especially wherein the second reactant 220 has a second chemical structure according to formula III:

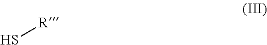

wherein R″ comprises the cargo.

FIG. 1A depicts an embodiment wherein R₀ is H, wherein R′is an alkyl group, wherein R″ is a thioester, wherein R₂ is an alkyl group, such as a methyl group, and wherein the second chemical structure is according to formula IIIA:

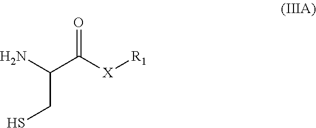

wherein X is O or NH, and wherein R1 comprises the cargo.

FIG. 1B schematically depicts an embodiment wherein Ro is selected from the group comprising halides, O-acyl groups, carbonate groups, sulfonate groups, and NR₃ groups, wherein R′is H.

In embodiments, the first radiation 140 may be selected from the range of 375-525 nm, such as the range of 400-525 nm. In particular, the first radiation 140 may comprise a first wavelength in the range of 375-525 nm, such as in the range of 400-525 nm. First radiation having such a wavelength may be particularly suitable to facilitate the photo-activated decarboxylation of the C-terminal end 111 of the peptide 110, which may be catalyzed by the first catalyst 130.

In embodiments, the first intermediate 150 may be exposed to a second environment 200 during the second stage. In particular, the first intermediate 150 may be exposed to a second solvent 201 during the second stage, especially wherein the second stage is executed in the second mixture comprising the second solvent 201. The second solvent 101 may especially comprise one or more of water, DMSO and DMF. In further embodiments, the second environment 200, especially the second mixture, comprises ≤1 ppm dissolved oxygen. In further embodiments, the first stage may be performed under inert gas, such as N₂ or Ar.

In embodiments, the peptide 110 may be exposed to a first environment 100 during the first stage. In particular, the peptide 110 may be exposed to a first solvent 101 during the first stage, especially wherein the first stage is executed in a first mixture comprising the first solvent 101. The first solvent 101 may especially comprise one or more of water, DMSO and DMF. In further embodiments, the first environment 100, especially the first mixture, comprises ≤1 ppm dissolved oxygen. In further embodiments, the second stage may be performed under inert gas, such as N₂ or Ar.

FIG. 1A-B further schematically depicts embodiments of the peptide-cargo conjugate 250.

In particular, FIG. 1A schematically depicts a peptide-cargo conjugate having a chemical structure according to formula IVA:

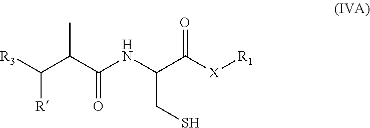

especially wherein R′ is an alkyl group, especially wherein R₁ comprises the cargo, and especially wherein R₃ refers to the peptide.

FIG. 1B schematically depicts a peptide-cargo conjugate having a chemical structure according to formula IVB:

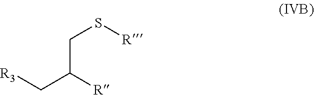

Especially wherein R″ is an electron withdrawing group comprising a functional group selected from the group consisting of an ester, a thioester, an amide, a ketone, a nitro, a sulfoxide, a sulfone, a phosphate ester, and an acylhydrazide, especially wherein R′″ refers to the cargo, and especially wherein R₃ comprises the peptide.

### Experiment 1—First Intermediate Comprising a Thioester Functional Group

First stage—2 μL (10.0 eq, 2.5 μmol, 0.435 mg) K₂HPO₄ stock solution (in H₂O) were added to a 4 mL dram vial and freeze dried. To the vial with the K2HPO4 residue 280 μL anhydrous DMSO, 50 μL (1.0 eq, 0.25 μmol) peptide 110 stock solution (in DMSO), 50 μL first catalyst 130 stock solution, particularly (0.12 eq, 0.03 μmol) Ir[dF(CF₃)ppy]₂(dtbbpy)PF₆ stock solution (in DMSO), 20 μL (10.0 eq, 2.5 μmol, 0.50 mg) first reactant stock solution (in DMSO) were added and the mixture was degassed for 10 minutes by sparging with nitrogen. Then the vial was flushed with argon, parafilmed and the mixture was irradiated for 16 hours with first radiation 140 using a blue LED (40 W, Kessil, approximately 4 cm distance).

In particular, Experiment 1 was performed with two different first reactants having chemical structure 1A, specifically wherein R′ comprises a methyl group, and wherein R₂ comprises either methylacetamide or methylpropanamide, i.e. with first reactants according to formula IA₁:

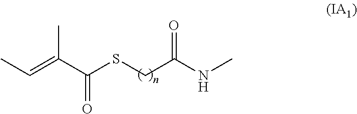

wherein n is either 1 or 2.

Next, to reduce, especially avoid, by-product formation, the sample was mixed with a thiol compound, specifically with 0.57 μL (20 eq, 5.0 μmol, 0.596 mg) 3-mercapto-N-methylpropanamide, or with 20 eq (5.0 μmol, 0.391 mg, 0.35 μL) 2-mercaptoethanol, and the mixture was frozen in liquid nitrogen and freeze-dried overnight. The different thiol compounds provided similar results.

Intermediate stage—The residue of the first stage was dissolved in 500 μL H₂O, using an ultrasonic bath, and divided into two parts. Each part was then extracted three times with 800 μL diethyl ether. Then both aqueous layers were dried from diethyl ether under argon stream, were combined, mixed and two portions of 200 μL (max. 0.1 μmol first intermediate 150) were transferred in two separate tubes. The separated fractions were closed with a perforated cap and freeze-dried.

Second stage—For the second stage, a native chemical ligation procedure was followed. Specifically, 286.6 mg (3 mmol) of denaturant guanidine hydrochloride was added to a separate tube and solubilized in 250 μL sodium phosphate buffer (100 mM Na₂HPO₄, pH 9). This mixture was then degassed with nitrogen for 10-15 minutes. In the following the reaction mixture was flushed with argon any time when a compound was added before mixing. The whole solution was then transferred to a tube which contains 16.82 mg (100 μmol) 4-mercaptophenylacetic acid and it was dissolved using an ultrasonic bath. 209 μL of this solution was then added to a tube with 3.01 mg (10.5 μmol) tris(2-carboxyethyl)phosphine hydrochloride and mixed until dissolution. To this mixture 20 μL 6 M sodium hydroxide was added, mixed and then the whole solution was transferred to the tube which contains the second reactant and mixed until dissolution, wherein the second reactant comprises a second peptide (2 eq, 0.2 μmol) with an N-terminal cysteine residue. Finally, the whole mixture was then transferred to the tube which contained one portion (max. 0.1 μmol) of the first intermediate 150 and was mixed. The pH was adjusted to pH 7.5-7.7 using 6 M NaOH. The reaction mixture was shacked at 37° C. for 2-3 days.

Thereby, a peptide-cargo conjugate was successfully obtained. In particular, as the cargo comprised a peptide, a fusion peptide was successfully obtained.

Second stage alternative—Alternatively, 573.2 mg (6 mmol) of denaturant guanidine hydrochloride and 500 μL sodium phosphate buffer was added to a vial. The whole solution was then transferred to the tube which contained 33.64 mg (200 μmol) 4-mercaptophenylacetic acid and it was dissolved using an ultrasonic bath. 840 μL of this solution was then added to a tube with 48.16 mg (168.0 μmol) TCEP and mixed until dissolution. This mixture was adjusted to pH 7.5 using 6 M NaOH. Then the ligation buffer was degassed for 25 minutes. 85.6 μL degassed ligation buffer was then transferred to the tube which contains the peptide with the N-terminal Cys residue, and mixed until dissolution. The whole mixture was then transferred to the tube which contained the first intermediate and mixed. Finally, the whole mixture was transferred into a 0.5 mL reaction tube, flushed with argon, closed and packed under argon inside a sealed glass vial and stirred 3 days at 37° C.

Thereby, a peptide-cargo conjugate was also successfully obtained. In particular, a (slightly) higher yield was obtained with the alternative procedure for the second stage.

### Experiment 2—First Intermediate Comprising a Michael Acceptor

Experiment 2 was performed using the following reactants: peptide 110; a K₂HPO₄ buffer pH 7.0, 100 mM; a first catalyst 130, particularly a riboflavin tetrabutyrate catalyst; glycerol 10% v/v; a first reactant 120, particularly methoxyester allyl acetate; DMF; and H₂O, Tris-HCl buffer pH 8.0, 20 mM, and a second reactant 220, especially ethyl 3-mercaptopropionate. In particular, two different peptides 110 were used for experiment 2: TRAP-6 (Thrombin Receptor Activator for Peptide 6; CAS 141136-83-6) and ACE I (Angiotensin I Converting Enzyme Inhibitor; CAS 58-82-2; NCBI accession number PODM76).

Preparation: A stock solution of the first catalyst was firstly prepared upon dissolving 2.46 mg in 1 mL of DMF. The first catalyst stock solution was separately degassed with N₂ for 10-15 min before adding it to the reaction solution. A stock solution of each peptide was also prepared and stored at −20° C. For TRAP-6:2.5 mg, 3.3 μmol in 668 μL DMF. For ACE I: 2.5 mg, 2.4 μmol in 472 μL DMF. A stock solution of the second reactant was prepared by dissolving 7.9 μL (8.38 mg) in 1 mL of DMF.

First stage—In a 4 mL vial equipped with a small stirring bar, 40 μL of K₂HPO₄ buffer (16.0 eq., 4.0 μmol) was added with a micropipette. Then, 200 μL of a glycerol solution in H₂O 10% v/v was added (final concentration around 4.5%). The first reactant 120 (0.4 μL=395 μg, 10.0 eq., 2.5 μmol) was added to the mixture together with DMF (30 μL) and milliQ H₂O (90 μL). Afterwards, 50 μL of peptide stock solution in DMF (1.0 eq, 0.25 μmol) was mixed in the reaction vial. At this point, the reaction vial was degassed with N₂ for 10-15 min in order to get rid of the O₂ that could interfere with the reaction outcome. After degassing, 40 μL of the first catalyst stock solution in DMF (0.6 eq., 0.098 mg, 0.150 μmol) was added in the vial under a flow of Ar. Before closing the vial, the screw cap was also flushed with Ar to avoid O₂ contamination. The vial was then covered with parafilm and the first radiation was provided using a blue led of 40 W for 8 h.

Intermediate stage—After 8 h irradiation, the crude reaction mixture was freeze-dried to remove the first solvent. The leftover was then dissolved in 500 μL of milliQ H₂O, transferred in a 2 mL Eppendorf and extracted 3 times with Et₂O.

Second stage—The crude reaction mixture previously dissolved in 500 μL of (milliQ) H₂O was split in two portions, each of 250 μL containing roughly 0.125 μmol of first intermediate 150. One portion was freeze dried to remove all the H₂O, and 90 μL of freshly prepared Tris-HCl buffer was added to the residue. Finally, 10 μL (5.0 eq., 0.625 μmol, 83.8 μg) of second reactant stock solution was added to the solution to trigger the reaction. Before starting stirring, the solution was flushed with Ar to remove O₂ that could interfere with the Thia-Michael addition by oxidizing and so inactivating the thiol to disulfide. The solution, in an Eppendorf tube, was then placed in a stirrer at 40° C. for 24 h.

Subsequently, MALDI-TOF analysis confirmed that for both peptides, peptide-cargo conjugates were successfully obtained.

In conclusion, C-terminal photo-decarboxylation alkylation with small acrylate linkers has been shown as a versatile way to introduce a reactive moiety onto amino acids and small peptides sequences. The experiments demonstrate how strong nucleophile like thiols can be engaged to undergo Michael addition with the first intermediate, facilitating site-and chemo-specific modifications.

### Experiment 3—Further First Reactants and Second Reactants

The method of the invention has further been applied with a variety of other first and second reactants, as summarized in the table below:

| # | First reactant | Second reactant | First stage | Second stage |
|---|---|---|---|---|
| 3.1 | 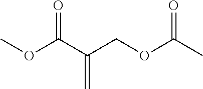 | Ethyl 3- mercaptopropionate | ++ | ++ |
| 3.2 | 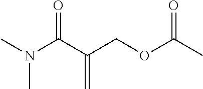 | Ethyl 3- mercaptopropionate | ++ | +/− |
| 3.3 | 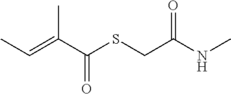 | Cys-Ala-Leu-Ala-Ala (SEQ ID NO: 1) | ++ | ++ |
| 3.4 | 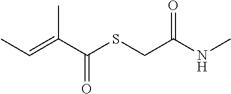 | Cys-Ala-Ala-Asn-Asp-Glu- Asn-Tyr-Ala-Leu-Ala-Ala (SEQ ID NO: 2) | ++ | ++ |
| 3.5 | 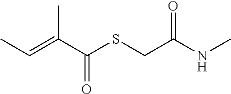 | Oxytocin (CAS 50-56-6) | ++ | ++ |
| 3.6 | 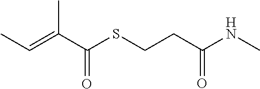 | Cys-Ala-Leu-Ala-Ala (SEQ ID NO: 1) | ++ | ++ |
| 3.7 | 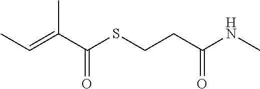 | Cys-Ala-Ala-Asn-Asp-Glu- Asn-Tyr-Ala-Leu-Ala-Ala (SEQ ID NO: 2) | ++ | ++ |
| 3.8 | 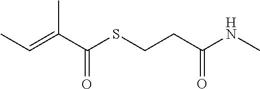 | Oxytocin (CAS 50-56-6) | ++ | ++ |
| 3.9 | 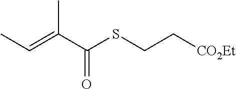 | Cys-Ala-Leu-Ala-Ala (SEQ ID NO: 1) | + | ++ |
| 3.10 | 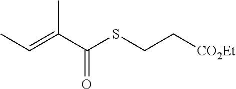 | Cys-Asp-Glu-Asn-Tyr-Ala- Leu-Ala-Ala (SEQ ID NO: 3) | + | ++ |
| 3.11 | 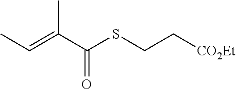 | Cys-Ala-Ala-Asn-Asp-Glu- Asn-Tyr-Ala-Leu-Ala-Ala (SEQ ID NO: 2) | + | ++ |
| 3.12 | 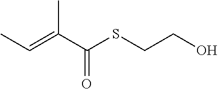 | — | +/− | N.D. |
| 3.13 | 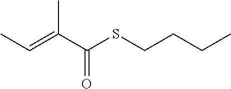 | — | +/− | N.D. |

With regards to the first stage and the second stage, “+/−” indicates a functional but poor result, “+” indicates a good result, “++” indicates an excellent result, and “N.D.” indicates not determined. Specifically, for experiment 3.2, the second stage proceeded slow, and the first reactants for experiments 3.2-3.8 demonstrated compatibility with peptides having a broad range of different C-terminal residues. For experiments 3.9-3.13, the stability of the first intermediate varied depending on the C-terminal residue of the peptide, despite experiments 3.9-3.11 performing excellent for a subset of C-terminal residues.

The term “plurality” refers to two or more. Furthermore, the terms “a plurality of” and “a number of” may be used interchangeably.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

The term “further embodiment” and similar terms may refer to an embodiment comprising the features of the previously discussed embodiment, but may also refer to an alternative embodiment.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method, respectively.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A method for providing a cargo to a C-terminal end of a peptide, the method comprising a first stage and a second stage, wherein the first stage comprises reacting the C-terminal end of the peptide with a first reactant in the presence of a first catalyst and first radiation to provide a first intermediate, wherein the first catalyst is configured to decarboxylate the C-terminal end of the peptide in the presence of the first radiation, and wherein the first reactant has a first chemical structure according to formula I: 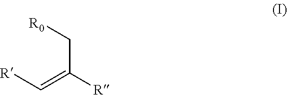
wherein R₀ is selected from the group consisting of H, halides, O-acyl groups, carbonate groups, sulfonate groups, and NR₃ groups, wherein each R is independently selected from H and alkyl groups, or wherein NR₃ comprises pyridinium or a derivative thereof;
wherein R′ is selected from the group consisting of H, aryl groups and alkyl groups;
wherein R″ is an electron withdrawing group comprising a functional group selected from the group consisting of an ester, a thioester, an amide, a ketone, a nitro, a sulfoxide, a sulfone, a phosphate ester, an acylhydrazide, a cyano group, and a trihalogenmethyl group;
and wherein the second stage comprises exposing the first intermediate to a second reactant, wherein the second reactant has a second chemical structure according to formula III: 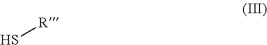
wherein R′″ comprises the cargo.

2. The method according to claim 1, wherein each R is independently selected from H and alkyl groups comprising 1-6 C atoms, and wherein R′is selected from the group consisting of H, aryl groups, and alkyl groups comprising 1-6 C atoms.

3. The method according to claim 1, wherein R₀ is H, wherein R′is an alkyl group, wherein R″ is a thioester, and wherein the second chemical structure is according to formula IIIA: 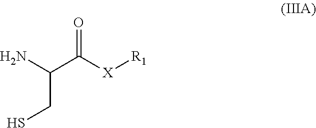
wherein X is O or NH, and wherein R1 comprises the cargo.

4. The method according to claim 1 wherein R₀ is selected from the group comprising halides, O-acyl groups, carbonate groups, sulfonate groups, and NR₃ groups, and wherein R′is H.

5. The method according to claim 1, wherein the first catalyst is selected from the group comprising riboflavin tetrabutyrate and Ir[dF(CF₃)ppy]₂(dtbbpy)PF₆.

6. The method according to claim 1, wherein the first radiation comprises a first wavelength in the range of 375-525 nm.

7. The method according to claim 1, wherein the first stage is executed in a first mixture comprising a first solvent, wherein the first solvent comprises one or more of water, DMSO and DMF.

8. The method according to claim 7, wherein the first mixture comprises ≤1 ppm dissolved oxygen.

9. The method according to claim 1, wherein the method further comprises an intermediate stage, wherein the intermediate stage comprises separating the first intermediate from the first reactant.

10. The method according to claim 1, wherein the second stage is performed in a degassed buffer under inert gas.

11. The method according to claim 1, wherein during the second stage the peptide is exposed to a second environment with a pH of 6-10.

12. The method according to claim 1, wherein the cargo comprises a second peptide.

13. The method according to claim 1, wherein the cargo comprises an anti-body.

14. A peptide-cargo conjugate obtainable using the method according to claim 1, wherein:
the peptide-cargo conjugate has a chemical structure according to formula IVA: 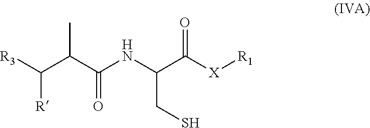
wherein X is O or NH, wherein R′ is an alkyl group, wherein R₁ comprises the cargo, and wherein R₃ refers to the peptide; or
the peptide-cargo conjugate has a chemical structure according to formula IVB: 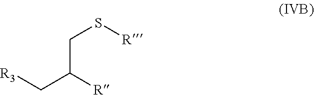
wherein R″ is an electron withdrawing group comprising a functional group selected from the group consisting of an ester, a thioester, an amide, a ketone, a nitro, a sulfoxide, a sulfone, a phosphate ester, and an acylhydrazide, wherein R′″ refers to the cargo, and wherein R₃ comprises the peptide.

15. The peptide-cargo conjugate of claim 14, wherein the peptide-cargo conjugate has the chemical structure according to formula IVA.

16. The peptide-cargo conjugate according to claim 14, wherein the peptide-cargo conjugate has the chemical structure according to formula IVB.

17. The peptide-cargo conjugate according to claim 14 for use as an anti-body drug conjugate.

18. A peptide array comprising the peptide-cargo conjugate according to claim 14.

19. Use of the peptide-cargo conjugate according to claim 16 for peptide sequencing.
